(19) United States
(12) Patent Application Publication
(10) Pub. No.: US 2009/0264835 A1
(43) Pub. Date: Oct. 22, 2009

(54) Detachable shaft medical retrieval device methods of use

(76) Inventor: James Francis Schuermann, Natick, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/457,934

(22) Filed: Jun. 25, 2009

Related U.S. Application Data
(62) Division of application No. 10/957,708, filed on Oct. 5, 2004.

(51) Int. Cl. A61B 1/00; A61M 25/00; A61F 2/01

(52) U.S. Cl. 604/266; 600/127; 606/200

(57) ABSTRACT

Embodiments of the invention are directed to a medical device and methods for immobilizing and retrieving material from a patient's body. The device may include a proximal portion extending substantially longitudinally and a detachably engaged to a distal portion capable of transforming between an expanded configuration and a substantially straight configuration.

### DESCRIPTION OF THE INVENTION

1. Field of the Invention

This invention relates to medical devices for medical treatment, immobilization, and retrieval of objects within anatomical lumens of the body. More particularly, the invention relates to retrieval methods and devices for stabilizing and retrieving objects found in the body.

2. Background of the Invention

Medical immobilization and retrieval devices may include devices for stabilizing and/or removing organic material (e.g., blood clots, tissue, and biological concretions such as urinary, biliary, and pancreatic stones) and inorganic material (e.g., components of a medical device or other foreign matter), which may obstruct or otherwise be present within a body's anatomical lumens. For example, concretions can develop in certain parts of the body, such as in the kidneys, pancreas, and gallbladder. Minimally invasive medical procedures generally involve causing limited trauma to the tissues of a patient, and can be used to dispose of problematic concretions. Lithotripsy and ureteroscopy, for example, are used to treat urinary calculi (e.g., kidney stones) in the ureter of patients.

Lithotripsy is a medical procedure that uses energy in various forms such as acoustic shock waves, pneumatic pulsation, electrical hydraulic shock waves, or laser beams to break up biological concretions such as urinary calculi (e.g., kidney stones). The force of the energy, when applied either extracorporeally or intracorporeally, usually in focused and continuous or successive bursts, divides a kidney stone into smaller fragments that may be extracted from the body or allowed to pass through urination. With the help of imaging tools such as transureteroscopic video technology and fluoroscopic imaging, the operator of the lithotripter device can monitor the progress of the medical procedure and terminate treatment when residual fragments are small enough to be voided or removed.

Intracorporeal fragmentation of urinary calculi can prove problematic in that stones and/or stone fragments in the ureter may become repositioned closer to and possibly migrate back toward the kidney, thereby requiring further medical intervention to prevent the aggravation of the patient's condition. Existing practices to control migration of stones during lithotripsy include reducing the energy or frequency of the lithotripsy, or reducing the amount or frequency of irrigation used during the procedure. Another known practice includes pushing the stone into the renal pelvis and undertaking another future procedure for its removal.

Alternatively, various devices may be deployed to control migration. For example, combined immobilization and retrieval devices, such as cone-shaped devices and baskets, may be deployed within a patient's body through the working channel of an ureteroscope. Once deployed past the stone, the immobilization device can act as a backstop to prevent upward migration of fragments resulting from a lithotripsy procedure. In order to, for example, maintain mobility of the ureteroscope for the proper viewing of the treatment site, it may be necessary to completely back off the ureteroscope from the body of the immobilization device maintained with the scope's working channel and reinsert the ureteroscope within the patient's body to reach the treatment site. This may require the use of an additional guidewire inserted to the treatment site. Removal and reinsertion of the ureteroscope also adds time to the procedure and may cause additional trauma to the patient.

Thus, it is desirable to have alternative methods of preventing upward migration of fragments, and extracting such fragments from the body.

### SUMMARY OF THE INVENTION

Embodiments of the present invention are directed to medical devices for immobilization and retrieval of objects within anatomical lumens of the body that obviate one or more of the limitations and disadvantages of prior immobilization and retrieval devices.

In one embodiment, the medical device includes a distal portion capable of transforming between an expanded configuration and a collapsed configuration and a shaft detachably engaged with a proximal end of the distal portion.

In various embodiments, the medical device may include one or more of the following additional features: wherein a proximal end of the distal portion includes a proximal hub detachably engaged with a distal portion of the shaft; wherein the distal portion comprises a shape-memory material; a sheath enclosing the distal portion when the distal portion is in the collapsed configuration, and wherein the distal portion transforms between the collapsed configuration and the expanded configuration when the distal portion is moved relative to the sheath; wherein the expanded configuration is a coil configuration and the collapsed configuration is a substantially straight configuration; wherein the coil configuration is a helical coil configuration; a polymer coating covering at least part of the distal portion; a sleeve configured to extend over the shaft; wherein the sleeve defines an internal lumen extending therethrough and terminating at an opening defined by a distal end of the sleeve; wherein a proximal end of the distal portion and the opening defined by the distal end of the sleeve are sized such that the opening does not accept the proximal end of the distal portion; wherein distal movement of the sleeve relative to the shaft forces disengagement of the shaft from the distal portion; wherein an outer diameter of the proximal end of the distal portion is greater than a diameter of the opening defined by the distal end of the sleeve; wherein an exterior of the proximal hub includes notches; wherein the shaft is configured to be disengaged and re-engaged with a proximal end of the distal portion; wherein the shaft is detachably engaged with the distal portion by a detachable engagement of male and female connecting parts; wherein the shaft is detachably engaged with the distal portion by a detachable ball and socket joint; wherein the shaft is detachably engaged by a friction fit engagement.

Another embodiment of the invention is directed to a method for immobilizing material in a body. The method includes providing a medical device comprising a distal portion and a shaft detachably engaged with a proximal end of the distal portion; inserting the medical device into an anatomical lumen of the body, with the distal portion of the medical device in a collapsed configuration; positioning the distal portion of the medical device beyond the material to be immobilized; transforming the distal portion of the medical device to the expanded configuration; and disengaging the shaft from the distal portion.

In various embodiments, the method may include one or more of the following additional features: performing a lithotripsy procedure on the material; wherein the medical device is inserted into an anatomical lumen of the body through a working channel of an endoscope; removing the shaft from the working channel of the endoscope after disengaging the shaft from the distal portion; irrigating of the lumen of the body through the working channel of the endoscope after removing the shaft from the working channel; prior to disengaging the shaft from the distal portion of the medical device, extending a sleeve over a proximal end of the shaft towards the distal portion of the medical device; wherein the sleeve defines an internal lumen extending therethrough and terminating at an opening defined by a distal end of the sleeve; wherein a proximal end of the distal portion and the opening defined by the distal end of the sleeve are sized such that the opening does not accept the proximal end of the distal portion; wherein the sleeve is extended over the shaft until the distal end of the sleeve contacts the proximal end of the distal portion; wherein disengaging the shaft from the distal portion includes moving the sleeve relative to the shaft; retrieving the immobilized material by removing the distal portion from the patient's body; inserting a grasper within the anatomical lumen of the body; grasping the distal portion; and retrieving the distal portion by removing the distal portion from the patient's body with the grasper; after disengaging the shaft from the distal portion, inserting a second shaft within the anatomical lumen of the body; engaging the distal portion with a distal end of the second shaft; and retrieving the distal portion by removing the distal portion from the patient's body upon proximal movement of the second shaft; wherein the second shaft is the shaft disengaged from the distal portion; wherein a proximal end of the distal portion includes a proximal hub configured to receive a distal portion of the shaft such that the distal portion of the shaft is detachably engaged with the proximal hub; wherein an exterior of the distal portion includes notches, and retrieving the distal portion includes removing the distal portion from the patient's body by engaging the notches with the grasper; wherein the medical device further comprises a sheath enclosing the distal portion when the distal portion is in the collapsed configuration, and wherein transforming the distal portion to the expanded configuration includes moving the distal portion relative to the sheath.

Another embodiment of the invention is directed to a method for immobilizing material in a body. The method includes inserting a catheter into an anatomical lumen of the body; positioning the distal portion of the catheter beyond the material to be immobilized within the lumen of the body; and injecting a substance from the distal portion of the catheter into the anatomical lumen of the body, the substance forming a gel within the lumen of the body.

In various embodiments, the method may include one or more of the following additional features: inserting the catheter into an anatomical lumen of the body through a working channel of an endoscope; removing the catheter from the working channel of the endoscope after injecting the substance; irrigating the lumen of the body through the working channel of the endoscope after removing the catheter from the working channel; performing a lithotripsy procedure on the material; retrieving the immobilized material by removing the gel and the immobilized material, after performing the lithotripsy procedure; inserting a grasper within the anatomical lumen of the body; grasping the gel; and retrieving the immobilized material and the gel by removing the grasper from the patient's body; wherein the catheter includes a radiopaque marker; wherein the gel substantially occludes the anatomical lumen distal to the material to be immobilized; wherein the substance exists in liquid form at temperatures below about body temperature and as a gel at temperatures about at and above body temperature.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a partial side cross-sectional view of a medical immobilization/retrieval device in an undeployed position according to an embodiment of the invention.

FIG. 2 is a side view of the medical immobilization/retrieval device of FIG. 1 in a deployed position.

FIGS. 3A and 3B illustrate a detachment procedure for the medical immobilization/retrieval device of FIG. 2, according to an embodiment of the invention.

FIG. 4 is a side view of the detached distal portion of the medical immobilization/retrieval device of FIG. 2.

FIG. 5 illustrates a procedure for retrieving a detached distal portion of a medical immobilization/retrieval device, according to an embodiment of the invention.

FIG. 6 is a side view of an alternative medical immobilization/retrieval device, according to an embodiment of the invention.

FIG. 7 illustrates a retrieval procedure for the medical immobilization/retrieval device of FIG. 6, according to an embodiment of the invention.

### DESCRIPTION OF THE EMBODIMENTS

Reference will now be made in detail to the present exemplary embodiments of the invention illustrated in the accompanying drawings. Wherever possible, the same reference numbers will be used throughout the drawings to refer to the same or like parts.

Coiled stabilization devices often include a unitary core element extending proximally from a coiled distal portion. The core element assists in moving the coiled distal portion forward relative to a sheath, for retraction of the coiled distal portion back within the lumen of a sheath (and into a substantially straightened collapsed shape), and for proximal movement of the coiled distal portion in order to ensnare the target object, for example, a kidney stone, within the coiled portion.

Embodiments of the current invention relate to medical immobilization/retrieval devices that, for example, may be inserted within the working channel of an endoscope during a medical procedure. Operative portions of the immobilization device can be detached from at least certain portions of the device that aid in deployment of the device. By detaching these portions, for example, the instrument is not maintained within the working channel of an endoscope during a medical procedure. Such a device would prevent the need for backing off and reinserting the ureteroscope, decreasing trauma to the lining of a patient's ureter and decreasing the duration and difficulty of the procedure. In addition, freeing the working channel of the ureteroscope can provide the additional advantage of increased mobility of the ureteroscope for viewing purposes. As a result, additional utility can be afforded by the unused channel, such as irrigation of the treatment site.

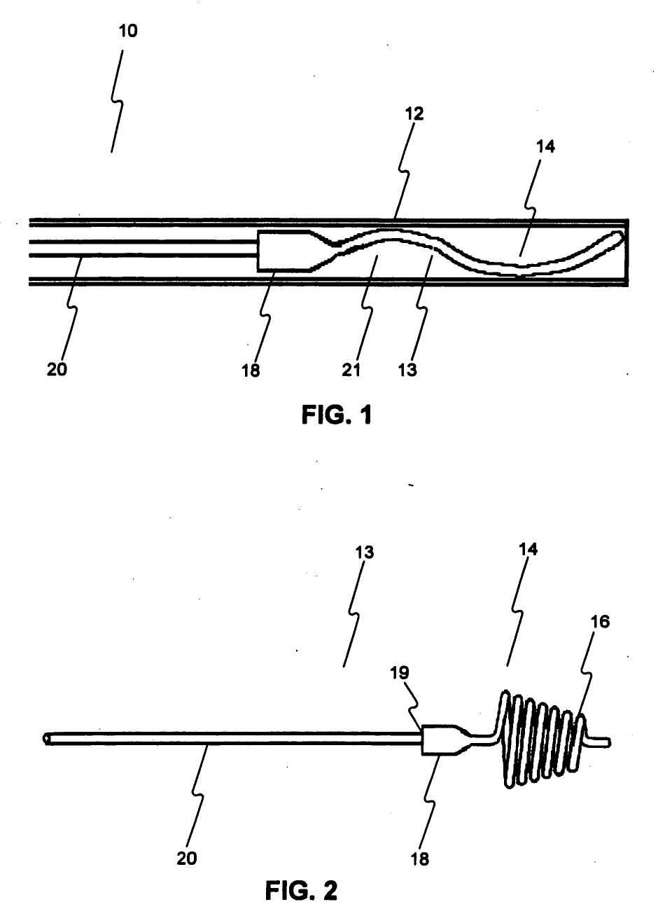

FIGS. 1 and 2, illustrate a medical device 10 having a sheath 12 and an inner immobilization device 13, according to an embodiment of the invention. Device 13 includes a shaft 20, a proximal hub 18 having a proximal-most face 19, and a coiled distal portion 14.

Coiled distal portion 14 can be made at least partially of a shape-memory material. Shape-memory material is a material that can be formed into a particular shape, retain that shape during resting conditions (e.g., when the shaped material is in free space or when external forces applied to the shaped material are insufficient to substantially deform the shape), be deformed into a second shape when subjected to a sufficiently strong external force, and revert substantially back to the initial shape when external forces are no longer applied. Examples of shape memory materials include synthetic plastics, stainless steel, and superelastic, metallic alloys of nickel/titanium (commonly referred to as nitinol), copper, cobalt, vanadium, chromium, iron, or the like.

As seen in FIG. 2, the coiled distal portion 14 can be wound to form a helical coil 16 in the absence of external forces. The helical coil 16 is adapted to taper from a larger diameter at a proximal end thereof to a smaller diameter at a distal end thereof, thereby resembling a helical cone shape. The sheath 12 and coiled distal portion 14 are movable relative to each other in order to achieve a first, relatively linear and substantially straight, collapsed state (FIG. 1) in which the coiled distal portion 14 is collapsed within an internal lumen 21 of the sheath 12 and a second state (FIG. 2) in which the coiled distal portion 14 extends from the distal end of the sheath 12 and expands to form a helical coil 16. A polymer coating may cover all or part of the coiled distal portion 14 for reducing the amount of friction between the surfaces of coiled distal portion 14 and sheath 12 during movement between expanded and collapsed states. In addition, the polymer coating may reduce friction between the coiled distal portion 14 and the lining of the ureteral wall into which the device is deployed.

The shaft 20 extends proximally from the proximal hub 18 to a point outside the internal lumen 21 of the sheath 12 where it may be manipulated by an operator. The shaft 20 is detachably engaged to the proximal hub 18 such that the shaft 20 may be disengaged from and, at least in certain embodiments reengaged to, the proximal hub 18 of the device 13. Upon deployment of the helical coil 16 by relative proximal movement of the sheath 12, the sheath 12 may be completely backed off from the shaft 20, leaving the immobilization device in the configuration shown in FIG. 2.

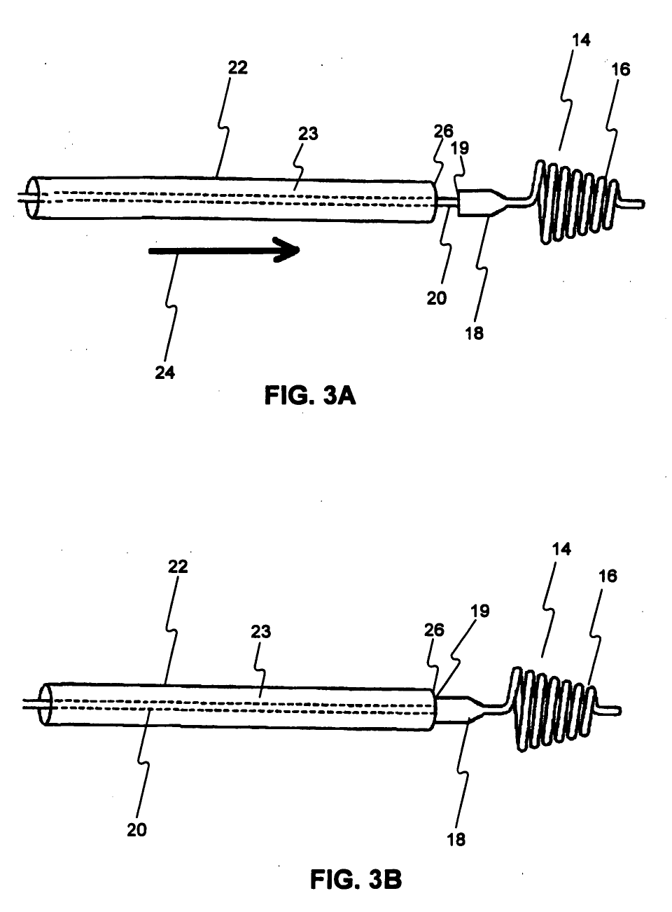

FIG. 3A illustrates the immobilization device 13 in the deployed configuration of FIG. 2 and further includes a release sleeve 22 tracked over the proximal end of the shaft 20. The release sleeve 22 includes a distal face 26 and an internal lumen 23. Arrow 24 of FIG. 3A designates that the sleeve 22 is tracked in a distal direction toward the proximal hub 18 after the immobilization device 13 is in the deployed configuration (FIG. 2).

One of both of the internal lumen 23 of release sleeve 22 and an opening defined by distal face 26 can be sized to have an inner diameter smaller than the outer diameter of the proximal hub 18. More broadly, the internal lumen 23 and/or the opening defined by the distal face 26 has a configuration (size, shape, dimensions, etc.) that does not accept proximal hub 18. The relative configurations, sizes, shapes, and/or dimensions between the opening at the distal face 26 of release sleeve 22 and the proximal face 19 of proximal hub 18 may be such that the distal face 26 contacts the proximal face 19 upon forward tracking of the release sleeve 22 to the proximal hub 18. Release sleeve 22 thereby will not continue to track over the proximal hub 18. Instead, the distal face of release sleeve 22 may abut the proximal face 19 of the proximal hub 18, preventing further forward movement of the release sleeve 22 relative to the immobilization device 13, as shown in FIG. 3B.

Upon continued movement of the release sleeve 22 relative to the immobilization device 13 in the direction of arrow 24 in FIG. 3A, the releasable shaft 20 can be detached from engagement with the proximal hub 18. Once the release sleeve 22 is tracked to the position of FIG. 3B, the operator can disengage shaft 20 from proximal hub 18 upon the application of sufficient pulling force at the proximal end of the releasable shaft 20. Upon the application of a sufficient proximally directed pulling force, while maintaining contact between the distal face 26 of release sleeve 22 and proximal face 19 of the proximal hub 18, shaft 20 can be disengaged from the proximal hub 18.

In other words, the relation between the shaft 20 and proximal hub 18 may be such that the shaft 20 is selectively or otherwise releasable from, and in certain embodiment re-engagable with, the proximal hub 18. The engagement between the shaft 20 and proximal hub 18 may be achieved, for example, by a male/female connection in which the distal portion of the shaft 20 is inserted and received within an opening in the proximal hub 18. The engagement may be realized, for example, by a ball and socket type connection, a friction fit engagement, a screw-like configuration or any other releasable engagement mechanisms known to one having ordinary skill in the art.

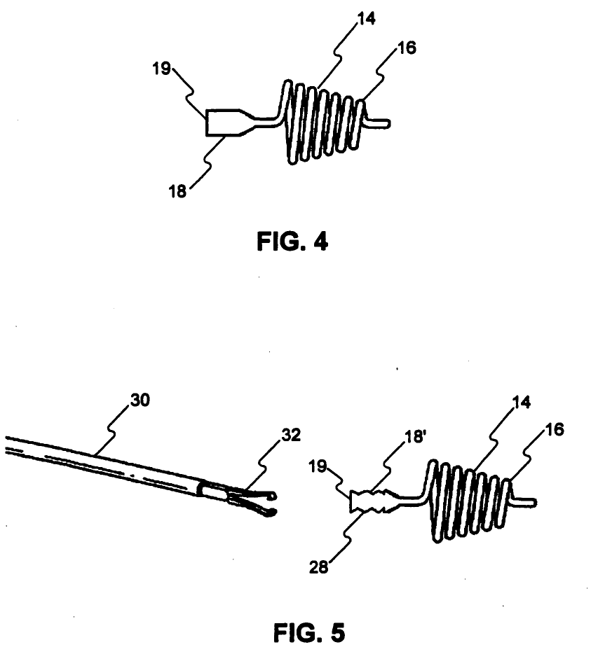

Referring to FIG. 4, the coiled distal portion 14 of immobilization device 13 is shown in a deployed configuration and detached from the shaft 20. FIG. 5 illustrates a surgical grasper 30 including grasping jaws 32 positioned at a treatment site near a deployed coiled distal portion 14. Grasper 30 may be used to retrieve portion 14 during a procedure. FIG. 5 shows a distal portion 14 that includes a proximal hub 18′ having notches 28 to facilitate the engagement by grasping jaws 32 with the proximal hub 18′.

Coiled stabilization devices, like those utilizing the design of a coiled distal portion 14, can be used to prevent the upward migration of stone fragments generated during a stone fragmentation procedure, and then safely and efficiently extract fragments from the body. For example, during a lithotripsy procedure, a coiled distal portion 14 can act as a backstop against any upward migration of stone fragments resulting from the procedure. The deployed distal portion 14 may remain substantially stationary due to the radial strength of the expanded helical coil 16 against the patient's internal anatomical lumen. Distal portion 14 may be then used to collect and remove the fragments from the body.

An embodiment of using device 10 includes positioning the device 10 of FIG. 1, including sheath 12, coiled distal portion 14 (in the substantially straightened collapsed shape of FIG. 1), and shaft 20 at an internal treatment site through the working channel of an ureteroscope. When the desired site is accessed by the distal end of the ureteroscope, the immobilization device 10 can be extended from the working channel of the scope. Once outside the working channel of the scope, the sheath 12 is retracted relative to the coiled distal portion 14 and shaft 20 in order to expand the distal portion 14 within an anatomical lumen and thereby deploy the helical coil 16 at a desired treatment site. Sheath 12 may then be completely backed off from the shaft 20 outside the body of the patient by control of the operator, leaving the distal portion 14 in the configuration of FIG. 2.

Referring to FIG. 3A, a release sleeve 22 can then be tracked over the proximal end of shaft 20 toward the deployed proximal hub 18 of immobilization device 10 in the direction of arrow 24. When the distal face 26 of release sleeve 22 contacts the proximal face 19 of the proximal hub 18, continued movement of the release sleeve 22 relative to the distal portion 14 in the direction of arrow 24 in FIG. 3A will result in the detachment of shaft 20 from engagement with the proximal hub 18. Other methods of releasing shaft 20 from hub 18 may be used, depending, for example, on the type of engagement between shaft 20 and hub 18.

Shaft 20 then can be removed from the working channel of the ureteroscope, leaving the helical coil 16 deployed at a desired point within a patient's anatomical lumen. At this point, the distal end of the ureteroscope is free to be more easily positioned relative to the helical coil 16 in order to maintain an image of the treatment site or, for example, to provide irrigation at a desired point within an anatomical lumen.

When a lithotripsy procedure is performed to fragment a stone into smaller fragments, the deployed helical coil 16 serves as a physical barrier or back-stop during the lithotripsy procedure to ensure that the smaller fragments do not migrate in an undesired direction, e.g., kidney stone fragments migrating back toward the kidney. The superelasticity of the helical coil coupled with its conical configuration provides a flexible barrier that is able to absorb the kinetic energy of the fragments produced when a laser or other energy is used to comminute or ablate the obstruction.

Once the lithotripsy procedure is complete, the operator can retrieve the deployed helical coil 16 in order to ensnare the remaining fragments. If the fragments are small enough to pass through the anatomical lumen, then the user can drag the fragments from the anatomical lumen and out of the body. Referring to FIG. 5, a surgical grasper 30 including grasping jaws 32 may be deployed into a patient's anatomical lumen in order to retrieve the deployed helical coil 16. An operator may engage the exterior of proximal hub 18′ with the grasping jaws 32 and drag the deployed helical coil 16, along with any ensnared fragments from the anatomical lumen.

Alternatively, the operator may reinsert the releasable shaft 20 in order to re-engage the proximal hub 18/18′ of the coiled distal portion 14. Upon re-engagement of the releasable shaft 20 with proximal hub 18,18′, the coiled distal portion 14 may be pulled proximally and out of the patient's anatomical lumen.

Regardless of whether the deployed coiled distal portion 14 is retrieved with the surgical grasper 30 or the releasable shaft 20, the coiled distal portion 14 may be retracted proximally back within the lumen of sheath 12 (through which shaft 20 or grasper 30 are inserted) and into the collapsed substantially straightened configuration of FIG. 1. In this configuration, the entire immobilization device can be removed from the anatomical lumen of the patient's body.

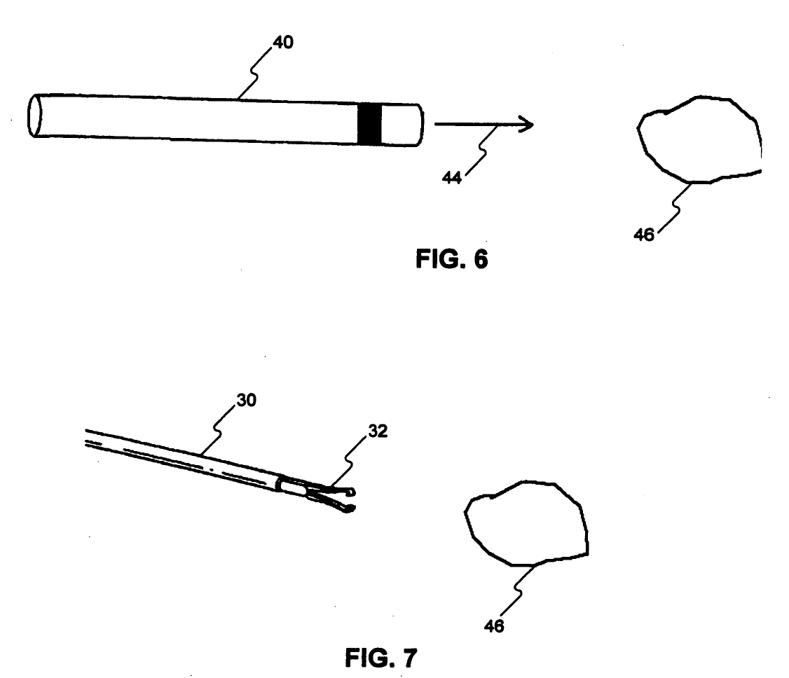

FIGS. 6-7 illustrate an alternative embodiment of an immobilization/retrieval device. The embodiment of FIGS. 6-7 includes a system and method of immobilizing and retrieving an object in the body of a patient. The method includes injecting a relatively soft gelatinous material into the body of the patient to serve as a physical barrier or back stop against the unwanted upward migration of objects to be removed, such as, for example urinary calculi and kidney stones. The gelatinous material may be formed from a material in flowable form when outside a patient's body and transforms to a gel form inside the patient's body. Such materials can include, for example, those disclosed for injection into a patient's body as set forth in U.S. Pat. No. 6,663,594, to Sahatjian et al., issued on Dec. 16, 2003, which is hereby incorporated by reference in its entirety (hereinafter the '594 patent). Such materials may include, for example, materials that transform from liquid form at a temperature below about body temperature and to a gel at temperatures about at or above body temperatures. One group of such materials are disclosed as LCST (lower critical solution temperature materials) materials in the '594 patent. Such materials also include, for example, one or more of a crosslinkable polymer, a gelatin material, or a block copolymer with reverse thermal gelation properties, such as those disclosed in the '594 patent. As also disclosed in the '594 patent, the material can include a first material formed of a crosslinkable polymer in a flowable form and a second material including a crosslinking agent. The first material may include one or more of an anionic crosslinkable polymer, a cationic crosslinkable polymer, or a non-ionically crosslinkable polymer. In other embodiments, the first material includes one or more of polyacrylic acids, polymethacrylic acid, alginic acid, pectinic acids, sodium alginate, potasium alginate, carboxy methyl cellulose, hyaluronic acid, heparin, carboxymethyl starch, carboxymethyl dextran, heparin sulfate, chondroitin sulfate, polyethylene amine, polysaccharides, chitosan, carboxymethyl ichitosan, cationic starch or salts thereof. The second material may include one or more of an anionic crosslinking ion, a cationic crosslinking ion, or a non-ionic crosslinking agent. In other embodiments of the method, the second material includes one or more of phosphate, citrate, borate, succinate, maleate, adipate, oxalate, calcium, magnesium, barium, strontium, boron, beryllium, aluminium, iron, copper, cobalt, lead, or silver ions. In still other embodiments of the method, the second material includes one or more of di-vinylsulfone, polycarboxylic acids, polycarboxylic anhydrides, polyamines, epihalohydrins, diepoxides, dialdehydes, diols, carboxylic acid halides, ketenes, polyfunctional aziridines, polyfunctional carbodiimides, polyisocyanate, glutaraldehyde, or polyfunctional crosslinkers including functional groups capable of reacting with organic acid groups.

FIGS. 6-7 illustrate a system and method for injecting and retrieving the stabilized gel and the objects that may entrap therein. In embodiments of the method, some of the fragments resulting from a lithotripsy procedure may remain at least partially within the gel and stabilized by the gel. These fragments can then be retrieved along with the gel. Other fragments may be captured by the gel and removed from the body as the gel is retrieved and swept out of the body.

Referring to FIG. 6, a catheter 40 may be positioned at an internal treatment site through the working channel of an ureteroscope. When the desired site is accessed by the distal end of the ureteroscope, the catheter may be advanced from the working channel and positioned with the help of imaging through the use of a radiopaque marker 42 at the distal end of the catheter 40. As represented by arrow 44, the operator may inject a material in flowable form beyond the desired treatment site. Any suitable known injection method may be used. The gel material will transform from a flowable form to a gel form inside the body of the patient, for example, by using material that remains in flowable form below about body temperature and forms a gel about at or above body temperature. Upon transformation, gel 46 may serve as a physical barrier or backstop against the unwanted upward migration of objects to be removed.

During a lithotripsy procedure, the gel 46 can be of a sufficiently solid form as to entrap, or otherwise stop movement of, any stone fragments migrating as a result of the lithotripsy procedure. In addition, the gel 46 can also be of a sufficiently solid form to be retrieved. For example, referring to FIG. 7, after a lithotrispy procedure, the gel 46 can be removed with the use of a grasper 30 having grasping jaws 32. Other suitable retrieval devices and methods may be used.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1.-17. (canceled)

18. A method for immobilizing material in a body comprising:
providing a medical device comprising a distal portion and a shaft detachably engaged with a proximal end of the distal portion;
inserting the medical device into an anatomical lumen of the body, with the distal portion of the medical device in a collapsed configuration;
positioning the distal portion of the medical device beyond the material to be immobilized;
transforming the distal portion of the medical device to an expanded configuration; and
disengaging the shaft from the distal portion.

19. The method of claim 18 further comprising performing a lithotripsy procedure on the material.

20. The method of claim 18 wherein the medical device is inserted into an anatomical lumen of the body through a working channel of an endoscope.

21. The method of claim 20 further comprising removing the shaft from the working channel of the endoscope after disengaging the shaft from the distal portion.

22. The method of claim 21 further comprising irrigating the lumen of the body through the working channel of the endoscope after removing the shaft from the working channel.

23. The method of claim 18 further comprising, prior to disengaging the shaft from the distal portion of the medical device, extending a sleeve over a proximal end of the shaft towards the distal portion of the medical device.

24. The method of claim 23 wherein the sleeve defines an internal lumen extending therethrough and terminating at an opening defined by a distal end of the sleeve.

25. The method of claim 24 wherein a proximal end of the distal portion and the opening defined by the distal end of the sleeve are sized such that the opening does not accept the proximal end of the distal portion.

26. The method of claim 24 wherein the sleeve is extended over the shaft until the distal end of the sleeve contacts the proximal end of the distal portion.

27. The method of claim 26 wherein disengaging the shaft from the distal portion includes moving the sleeve relative to the shaft.

28. The method of claim 18 further comprising retrieving the immobilized material by removing the distal portion from the body.

29. The method of claim 18 further comprising:
inserting a grasper within the anatomical lumen of the body;
grasping the distal portion; and
retrieving the distal portion by removing the distal portion from the body with the grasper.

30. The method of claim 18 further comprising:
after disengaging the shaft from the distal portion, inserting a second shaft within the anatomical lumen of the body;
engaging the distal portion with a distal end of the second shaft; and
retrieving the distal portion by removing the distal portion from the body upon proximal movement of the second shaft.

31. The method of claim 30 wherein the second shaft is the shaft disengaged from the distal portion.

32. The method of claim 18 wherein a proximal end of the distal portion includes a proximal hub configured to receive a distal portion of the shaft such that the distal portion of the shaft is detachably engaged with the proximal hub.

33. The method of claim 29 wherein an exterior of the distal portion includes notches, and retrieving the distal portion includes removing the distal portion from the body by engaging the notches with the grasper.

34. The method of claim 18 wherein the medical device further comprises a sheath enclosing the distal portion when the distal portion is in the collapsed configuration, and wherein transforming the distal portion to the expanded configuration includes moving the distal portion relative to the sheath.

35. A method for immobilizing material in a body comprising:
inserting a catheter into an anatomical lumen of the body;
positioning the distal portion of the catheter beyond the material to be immobilized within the lumen of the body; and
injecting a substance from the distal portion of the catheter into the anatomical lumen of the body, the substance forming a gel within the lumen of the body.

36. The method of claim 35 further comprising inserting the catheter into an anatomical lumen of the body through a working channel of an endoscope.

37. The method of claim 36 further comprising removing the catheter from the working channel of the endoscope after injecting the substance.

38. The method of claim 37 further comprising irrigating the lumen of the body through the working channel of the endoscope after removing the catheter from the working channel.

39. The method of claim 35 further comprising performing a lithotripsy procedure on the material.

40. The method of claim 39 further comprising retrieving the immobilized material by removing the gel and the immobilized material, after performing the lithotripsy procedure.

41. The method of claim 35 further comprising:
inserting a grasper within the anatomical lumen of the body;
grasping the gel; and
retrieving the immobilized material and the gel by removing the grasper from the body.

42. The method of claim 35 wherein the catheter includes a radiopaque marker.

43. The method of claim 35 wherein the gel substantially occludes the anatomical lumen distal to the material to be immobilized.

44. The method of claim 35 wherein the substance exists in liquid form at temperatures below about body temperature and as a gel at temperatures about at and above body temperature.

45. A method for immobilizing material in a body comprising:
inserting a medical device into an anatomical lumen of the body, the medical device comprising a distal portion and a shaft detachably engaged with the distal portion;
positioning the distal portion of the medical device beyond the material to be immobilized;
disengaging the shaft from the distal portion; and
retrieving the material from the body by re-engaging the shaft with the distal portion and removing the distal portion from the body.

46. The method of claim 45 wherein inserting the medical device into the anatomical lumen of the body further includes inserting the distal portion in a collapsed position.

47. The method of claim 45 further including, after positioning the distal portion of the medical device beyond the material, transforming the distal portion of the medical device to an expanded configuration.

48. The method of claim 45 further including performing a lithotripsy procedure on the material.

49. The method of claim 45 wherein re-engaging the shaft with the distal portion includes inserting the shaft within an opening in a proximal end of the distal portion.

50. The method of claim 45 wherein re-engaging the shaft with the distal portion includes inserting a ball of the shaft into a socket of a proximal end of the distal portion.

51. The method of claim 45 wherein retrieving the material from the body further includes proximally pulling the shaft and distal portion out of the anatomical lumen of the body.
